(19) United States
(12) Patent Application Publication
(10) Pub. No.: US 2014/0066444 A1
(43) Pub. Date: Mar. 6, 2014

(54) Method of Treating Cancer

(71) Applicants: David C. Smith, Ann Arbor, MI (US); Maha Hussain, Ann Arbor, MI (US)

(72) Inventors: David C. Smith, Ann Arbor, MI (US); Maha Hussain, Ann Arbor, MI (US)

(73) Assignee: Exelixis, Inc., South San Francisco, CA (US)

(21) Appl. No.: 13/876,318

(22) PCT Filed: Sep. 26, 2011
(86) PCT No.: PCT/US2011/053240

§ 371 Date: Oct. 25, 2013

Related U.S. Application Data
(60) Provisional application No. 61/386,975, filed on Sep. 27, 2010.

(51) Int. Cl. A61K 31/5377

(52) U.S. Cl. CPC A61K 31/5377
USPC 514/235.2

(57) ABSTRACT

This invention is directed to the treatment of cancer, particularly castration-resistant prostate cancer and osteoblastic bone metastases, with a dual inhibitor of MET and VEGF.

### CROSS-REFERENCE TO RELATED APPLICATIONS

This application claims the benefit of priority to U.S. Provisional Application No. 61/386,975, filed Sep. 27, 2010, which is incorporated herein by reference.

### FIELD OF THE INVENTION

This invention is directed to the treatment of cancer, particularly castration-resistant prostate cancer and osteoblastic bone metastases, with a dual inhibitor of MET and VEGF.

### BACKGROUND OF THE INVENTION

Castration-Resistant Prostate Cancer (CRPC) is a leading cause of cancer-related death in men. Despite progress in systemic therapy for CRPC, improvements in survival are modest, and virtually all patients succumb to this disease with a median survival of about 2 years. The primary cause of morbidity and mortality in CRPC is metastasis to the bone, which occurs in about 90% of cases.

Metastasis to bone is a complex process involving interactions between cancer cells and components of the bone microenvironment including osteoblasts, osteoclasts, and endothelial cells. Bone metastases cause local disruption of normal bone remodeling, and lesions generally show a propensity for either osteoblastic (bone-forming) or osteolytic (bone-resorbing) activity. Although most CRPC patients with bone metastases display features of both types of lesions, prostate cancer bone metastases are often osteoblastic, with abnormal deposition of unstructured bone accompanied by increased skeletal fractures, spinal cord compression, and severe bone pain.

The receptor tyrosine kinase MET plays important roles in cell motility, proliferation, and survival, and has been shown to be a key factor in tumor angiogenesis, invasiveness, and metastasis. Prominent expression of MET has been observed in primary and metastatic prostate carcinomas, with evidence for higher levels of expression in bone metastases compared to lymph node metastases or primary tumors.

Vascular endothelial growth factor (VEGF) and its receptors on endothelial cells are widely accepted as key mediators in the process of tumor angiogenesis. In prostate cancer, elevated VEGF in either plasma or urine is associated with shorter overall survival. VEGF may also play a role in activating the MET pathway in tumor cells by binding to neuropilin-1, which is frequently upregulated in prostate cancer and appears to activate MET in a co-receptor complex. Agents targeting the VEGF signaling pathway have demonstrated some activity in patients with CRPC.

Thus, a need remains for methods of treating prostate cancer including CRPC and the associated osteoblastic bone metastases.

### SUMMARY OF THE INVENTION

These and other needs are met by the present invention which is directed to a method for treating bone cancer, prostate cancer, or bone cancer associated with prostate cancer. The method comprises administering a therapeutically effective amount of a compound that modulates both MET and VEGF to a patient in need of such treatment. In one embodiment, the bone cancer is osteoblastic bone metastases. In a further embodiment, the prostate cancer is CRPC. In a further embodiment, the bone cancer is osteoblastic bone metastases associated with CRPC.

In one aspect, the present invention is directed to a method for treating osteoblastic bone metastases CRPC, or osteoblastic bone metastases associated with CRPC, comprising administering a therapeutically effective amount of a compound that dually modulates MET and VEGF to a patient in need of such treatment.

In one embodiment of this and other aspects, the dual acting MET/VEGF inhibitor is a compound of Formula I

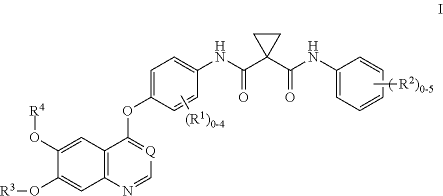

or a pharmaceutically acceptable salt thereof, wherein:

R¹ is halo;

R² is halo;

R³ is (C₁-C₆)alkyl substituted with heterocycloalkyl;

R⁴ is (C₁-C₆)alkyl; and

Q is CH or N.

In another embodiment, the compound of Formula I is the compound of Formula Ia:

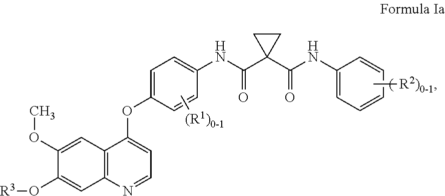

or a pharmaceutically acceptable salt thereof, wherein:

R¹ is halo;

R² is halo; and

R³ is (C₁-C₆)alkyl substituted with heterocycloalkyl.

In another embodiment, the compound of Formula I is Compound 1:

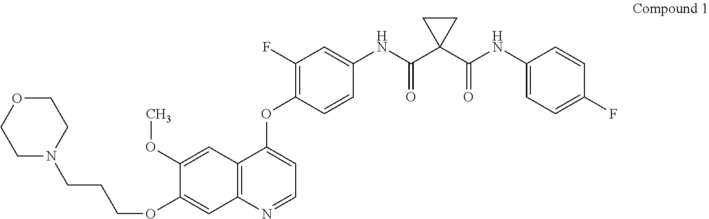 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt thereof. Compound 1 is known as is N-[3-fluoro-4-([6-(methyloxy)-7-[(3-morpholin-4-ylpropyl)oxy]quinolin-4-yl]oxy)phenyl]-N′-(4-fluorophenypcyclopropane-1,1-dicarboxamide. WO 2005-030140 describes the synthesis of Compound 1 (Examples 25, 30, 36, 42, 43 and 44) and also discloses the therapeutic activity of this molecule to inhibit, regulate and/or modulate the signal transduction of kinases, (Assays, Table 4, entry 312). Compound 1 has been measured to have a c-Met IC₅₀ value of about 0.6 nanomolar (nM). PCT/US09/064,341, which claims priority to U.S. provisional application 61/199,088, filed Nov. 13, 2008, describes a scaled-up synthesis of Compound 1.

In another embodiment, the compound of Formula I, Ia, or Compound 1 is administered as a pharmaceutical composition comprising a pharmaceutically acceptable additive, diluent, or excipient.

In another aspect, the invention provides a method for treating osteoblastic bone metastases associated with CRPC, comprising administering a therapeutically effective amount of a pharmaceutical composition comprising Compound of Formula I or the malate salt of Compound of Formula I or another pharmaceutically acceptable salt of Compound of Formula I, to a patient in need of such treatment. In a specific embodiment, the Compound of Formula I is Compound 1.

In another aspect, the invention provides a method for reducing or stabilizing metastatic bone lesions associated with CRPC, comprising administering a therapeutically effective amount of a pharmaceutical composition comprising Compound of Formula I, Ia or the malate salt of Compound of Formula I or another pharmaceutically acceptable salt of Compound of Formula I, to a patient in need of such treatment. In a specific embodiment, the Compound of Formula I is Compound 1.

In another aspect, the invention provides a method for reducing bone pain due to metastatic bone lesions associated with CRPC, comprising administering a therapeutically effective amount of a pharmaceutical composition comprising Compound of Formula I or the malate salt of Compound of Formula I or another pharmaceutically acceptable salt of Compound of Formula I, to a patient in need of such treatment. In a specific embodiment, the Compound of Formula I is Compound 1.

In another aspect, the invention provides a method for treating or minimizing bone pain due to metastatic bone lesions associated with CRPC, comprising administering a therapeutically effective amount of a pharmaceutical composition comprising Compound of Formula I or the malate salt of Compound of Formula I or another pharmaceutically acceptable salt of Compound of Formula I, to a patient in need of such treatment. In a specific embodiment, the Compound of Formula I is Compound 1.

In another aspect, the invention provides a method for strengthening bones in patients with metastatic bone lesions associated with CRPC, comprising administering a therapeutically effective amount of a pharmaceutical composition comprising Compound of Formula I or the malate salt of Compound of Formula I or another pharmaceutically acceptable salt of Compound of Formula I, to a patient in need of such treatment. In a specific embodiment, the Compound of Formula I is Compound 1. Bone strengthening can occur when the disruption in normal bone remodeling due to bone metastases is minimized, for instance by administering a Compound of Formula I as provided herein.

In another aspect, the invention provides a method for preventing osteoblastic bone metastases associated with CRPC, comprising administering a therapeutically effective amount of a pharmaceutical composition comprising Compound of Formula I or the malate salt of Compound of Formula I or another pharmaceutically acceptable salt of Compound of Formula I, to a patient in need of such treatment. In a specific embodiment, the Compound of Formula I is Compound 1.

In another aspect, the invention provides a method for preventing bone metastases in patients with prostate cancer who are castration resistant but have not yet advanced to metastatic disease, comprising administering a therapeutically effective amount of a pharmaceutical composition comprising Compound of Formula I or the malate salt of Compound of Formula I or another pharmaceutically acceptable salt of Compound of Formula I, to a patient in need of such treatment. In a specific embodiment, the Compound of Formula I is Compound 1.

In another aspect, the invention provides a method for extending the overall survival in patients with CRPC, comprising administering a therapeutically effective amount of a pharmaceutical composition comprising Compound of Formula I or the malate salt of Compound of Formula I or another pharmaceutically acceptable salt of Compound of Formula I, to a patient in need of such treatment.

In these and other aspects, the ability of the compound of Formula Ito treat, ameliorate, or reduce the severity of bone metastases can be determined both qualitatively and quantitatively using various physiological markers, such as circulating tumor cell (CTC) counts and imaging technologies. The imaging technologies include positron emission tomography (PET) or computerized tomography (CT) and magnetic resonance imaging. By using these imaging techniques, it is possible to monitor and quantify the reduction in tumor size and the reduction in the number and size of bone lesions in response to treatment with the compound of Formula I.

In these and other aspects, shrinkage of soft tissue and visceral lesions may be observed when the compound of Formula I is administered to patients with CRPC. Moreover, administration of the compound of Formula I leads to increases in hemoglobin concentration in patients CRPC patients with anemia.

### DETAILED DESCRIPTION OF THE INVENTION

### Abbreviations and Definitions

The following abbreviations and terms have the indicated meanings throughout:

| Abbreviation | Meaning |
|---|---|
| Ac | Acetyl |
| Br | Broad |
| ° C. | degrees Celsius |
| c- | Cyclo |
| CBZ | CarboBenZoxy = benzyloxycarbonyl |
| d | Doublet |
| dd | doublet of doublet |
| dt | doublet of triplet |
| DCM | Dichloromethane |
| DME | 1,2-dimethoxyethane |
| DMF | N,N-Dimethylformamide |
| DMSO | dimethyl sulfoxide |
| Dppf | 1,1′-bis(diphenylphosphano)ferrocene |
| EI | Electron Impact ionization |
| G | gram(s) |
| h or hr | hour(s) |
| HPLC | high pressure liquid chromatography |
| L | liter(s) |
| M | molar or molarity |
| m | Multiplet |
| Mg | milligram(s) |
| MHz | megahertz (frequency) |
| Min | minute(s) |
| mL | milliliter(s) |
| μL | microliter(s) |
| μM | Micromole(s) or micromolar |
| mM | Millimolar |
| Mmol | millimole(s) |
| Mol | mole(s) |
| MS | mass spectral analysis |
| N | normal or normality |
| nM | Nanomolar |
| NMR | nuclear magnetic resonance spectroscopy |
| q | Quartet |
| RT | Room temperature |
| s | Singlet |
| t or tr | Triplet |
| TFA | trifluoroacetic acid |
| THF | Tetrahydrofuran |
| TLC | thin layer chromatography |

The symbol “—” means a single bond, “═” means a double bond.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

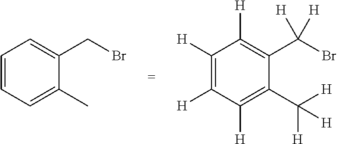

If a group “R” is depicted as “floating” on a ring system, as for example in the formula:

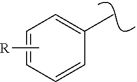

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the formulae:

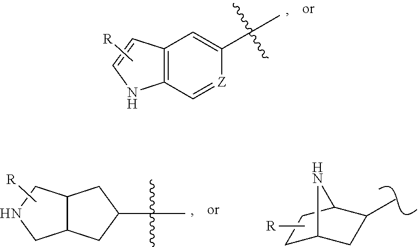

then, unless otherwise defined, a substituent “R” may reside on any atom of the fused ring system, assuming replacement of a depicted hydrogen (for example the —NH— in the formula above), implied hydrogen (for example as in the formula above, where the hydrogens are not shown but understood to be present), or expressly defined hydrogen (for example where in the formula above, “Z” equals ═CH—) from one of the ring atoms, so long as a stable structure is formed. In the example depicted, the “R” group may reside on either the 5-membered or the 6-membered ring of the fused ring system. In the formula depicted above, when y is 2 for example, then the two “R's” may reside on any two atoms of the ring system, again assuming each replaces a depicted, implied, or expressly defined hydrogen on the ring.

When a group “R” is depicted as existing on a ring system containing saturated carbons, as for example in the formula:

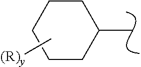

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group; there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

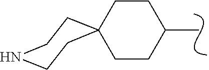

“Halogen” or “halo” refers to fluorine, chlorine, bromine or iodine.

“Yield” for each of the reactions described herein is expressed as a percentage of the theoretical yield.

“Patient” for the purposes of the present invention includes humans and other animals, particularly mammals, and other organisms. Thus the methods are applicable to both human therapy and veterinary applications. In another embodiment the patient is a mammal, and in another embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, malic acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Therapeutically effective amount” is an amount of a compound of the invention, that when administered to a patient, ameliorates a symptom of the disease. A therapeutically effective amount is intended to include an amount of a compound alone or in combination with other active ingredients effective to modulate c-Met, and/or VEGFR, or effective to treat or prevent cancer. The amount of a compound of the invention which constitutes a “therapeutically effective amount” will vary depending on the compound, the disease state and its severity, the age of the patient to be treated, and the like. The therapeutically effective amount can be determined by one of ordinary skill in the art having regard to their knowledge and to this disclosure.

“Treating” or “treatment” of a disease, disorder, or syndrome, as used herein, includes (i) preventing the disease, disorder, or syndrome from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experience.

### Embodiments

In one embodiment, the compound of Formula I is the compound of Formula I(a):

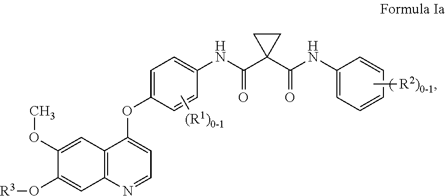

or a pharmaceutically acceptable salt thereof, wherein:

R¹ is halo;

R² is halo; and

R³ is (C₁-C₆)alkyl substituted with heterocycloalkyl.

In another embodiment, the compound of Formula I is Compound 1.

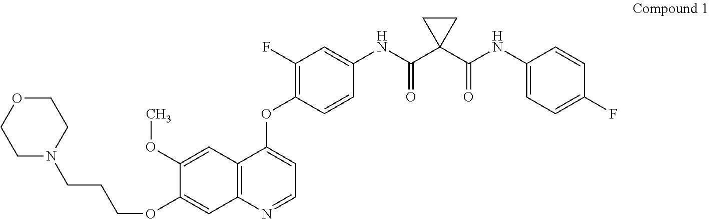 [SMILES: COc1cc2c(Oc3ccc(NC(=O)C4(C(=O)Nc5ccc(F)cc5)CC4)cc3F)ccnc2cc1OCCCN1CCOCC1]

In other embodiments, the compound of Formula I, Ia, or Compound 1, or a pharmaceutically acceptable salt thereof, is administered as a pharmaceutical composition, wherein the pharmaceutical composition additionally comprises a pharmaceutically acceptable carrier, excipient, or diluent. In a specific embodiment, the Compound of Formula I is Compound 1.

The compound of Formula I, Formula Ia and Compound I, as described herein, includes both the recited compounds as well as individual isomers and mixtures of isomers. In each instance, the compound of Formula I includes the pharmaceutically acceptable salts, hydrates, and/or solvates of the recited compounds and any individual isomers or mixture of isomers thereof.

In another embodiment, the invention is directed to a method for ameliorating the symptoms of osteoblastic bone metastases, comprising administering to a patient in need of such treatment a therapeutically effective amount of a compound of Formula I in any of the embodiments disclosed herein. In a specific embodiment, the Compound of Formula I is Compound 1.

In another embodiment, the compound of Formula I is administered post-taxotere treatment. In a specific embodiment, the Compound of Formula I is Compound 1.

In another embodiment, the compound of Formula I is as effective or more effective than mitoxantrone plus prednisone. In a specific embodiment, the Compound of Formula I is Compound 1.

In another embodiment, the Compound of Formula I, Ia, or Compound 1 or a pharmaceutically acceptable salt thereof is administered orally once daily as a tablet or capsule.

In another embodiment, Compound 1 is administered orally as a capsule or tablet.

In another embodiment, Compound 1 is administered orally once daily as a capsule or tablet containing up to 100 mg of Compound 1.

In another embodiment, Compound 1 is administered orally once daily as a capsule or tablet containing 100 mg of Compound 1.

In another embodiment, Compound 1 is administered orally once daily as a capsule or tablet containing 95 mg of Compound 1.

In another embodiment, Compound 1 is administered orally once daily as a capsule or tablet containing 90 mg of Compound 1.

In another embodiment, Compound 1 is administered orally once daily as a capsule or tablet containing 85 mg of Compound 1.

In another embodiment, Compound 1 is administered orally once daily as a capsule or tablet containing 80 mg of Compound 1.

In another embodiment, Compound 1 is administered orally once daily as a capsule or tablet containing 75 mg of Compound 1.

In another embodiment, Compound 1 is administered orally once daily as a capsule or tablet containing 70 mg of Compound 1.

In another embodiment, Compound 1 is administered orally once daily as a capsule or tablet containing 65 mg of Compound 1.

In another embodiment, Compound 1 is administered orally once daily as a capsule or tablet containing 60 mg of Compound 1.

In another embodiment, Compound 1 is administered orally once daily as a capsule or tablet containing 55 mg of Compound 1.

In another embodiment, Compound 1 is administered orally once daily as a capsule or tablet containing 50 mg of Compound I.

In another embodiment, Compound 1 is administered orally once daily as a capsule or tablet containing 45 mg of Compound 1.

In another embodiment, Compound 1 is administered orally once daily as a capsule or tablet containing 40 mg of Compound 1.

In another embodiment, Compound 1 is administered orally once daily as a capsule or tablet containing 30 mg of Compound 1.

In another embodiment, Compound 1 is administered orally once daily as a capsule or tablet containing 25 mg of Compound 1.

In another embodiment, Compound 1 is administered orally once daily as a capsule or tablet containing 20 mg of Compound 1.

In another embodiment, Compound 1 is administered orally once daily as a capsule or tablet containing 15 mg of Compound 1.

In another embodiment, Compound 1 is administered orally once daily as a capsule or tablet containing 10 mg of Compound 1.

In another embodiment, Compound 1 is administered orally once daily as a capsule or tablet containing 5 mg of Compound 1.

### Administration

Administration of the compound of Formula I, Ia, or Compound 1 or a pharmaceutically acceptable salt thereof, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin dosages (which can be in capsules or tablets), powders, solutions, suspensions, or aerosols, or the like, specifically in unit dosage forms suitable for simple administration of precise dosages.

The compositions will include a conventional pharmaceutical carrier or excipient and a compound of Formula I, Ia, or Compound 1 as the/an active agent, and, in addition, may include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the compound of Formula I, Ia, or Compound 1 may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated. In one embodiment, the oral dosage form is in the form of a capsule. In another embodiment, the oral dosage form is in the form of a tablet.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the compound of Formula I, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administration are, for example, suppositories that can be prepared by mixing the compound of Formula I with, for example, suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of the compound of Formula I include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this disclosure.

Compressed gases may be used to disperse the compound of Formula I in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of Formula I, or a pharmaceutically acceptable salt thereof, and 99% to 1% by weight of a suitable pharmaceutical excipient. In one example, the composition will be between about 5% and about 75% by weight of a compound(s) of Formula I, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of Formula I, or a pharmaceutically acceptable salt thereof, for treatment of a disease-state in accordance with the teachings of this disclosure.

The compounds of this disclosure, or their pharmaceutically acceptable salts or solvates, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compound of Formula I can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

In other embodiments, the compound of Formula I, IA, or Compound 1 or Compound 1, can be administered to the patient concurrently with other cancer treatments. Such treatments include other cancer chemotherapeutics, hormone replacement therapy, radiation therapy, or immunotherapy, among others. The choice of other therapy depends on a number of factors including the metabolic stability and length of action of the compound, the age, body weight, general health, sex, diet, mode and time of administration, rate of excretion, drug combination, the severity of the particular disease-states, and the host undergoing therapy.

### Preparation of Compound 1

Compound 1 was prepared as provided in Scheme 1 and the accompanying experimental examples.

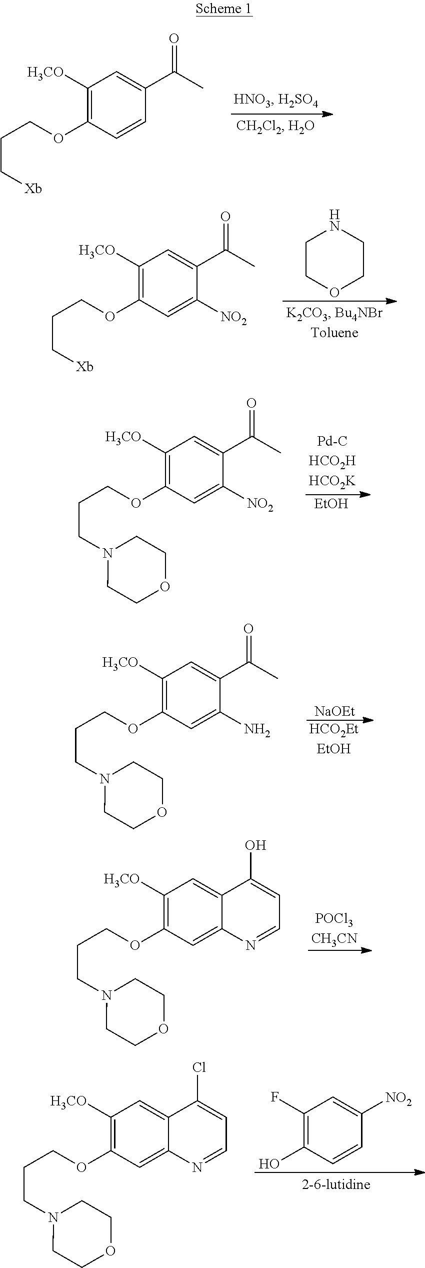

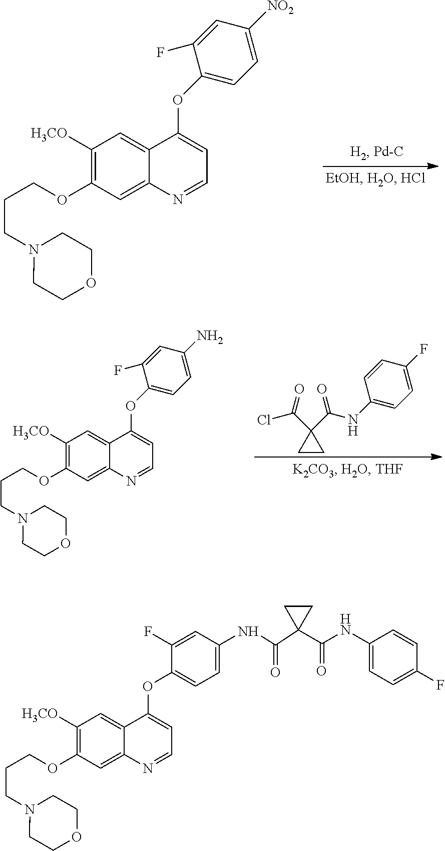 [SMILES: COc1cc2c(Oc3ccc([N+](=O)[O-])cc3F)ccnc2cc1OCCCN1CCOCC1 ; COc1cc2c(Oc3ccc(N)cc3F)ccnc2cc1OCCCN1CCOCC1 ; O=C(Cl)C1(C(=O)Nc2ccc(F)cc2)CC1 ; COc1cc2c(Oc3ccc(NC(=O)C4(C(=O)Nc5ccc(F)cc5)CC4)cc3F)ccnc2cc1OCCCN1CCOCC1]

In Scheme 1, Xb is Br or Cl. For the names of the intermediates described within the description of Scheme 1 below, Xb is referred to as halo, wherein this halo group for these intermediates is meant to mean either Br or Cl.

### Preparation of 1-[5 methoxy-4 (3-halo propoxy)-2 nitro-phenyl]-ethanone

Water (70 L) was charged to the solution of 1-[4-(3-halo propoxy)-3-methoxy phenyl]ethanone (both the bromo and the chloro compound are commercially available). The solution was cooled to approximately 4° C. Concentrated sulfuric acid (129.5 kg) was added at a rate such that the batch temperature did not exceed approximately 18° C. The resulting solution was cooled to approximately 5° C. and 70 percent nitric acid (75.8 kg) was added at a rate such that the batch temperature did not exceed approximately 10° C. Methylene chloride, water and ice were charged to a separate reactor. The acidic reaction mixture was then added into this mixture. The methylene chloride layer was separated and the aqueous layer was back extracted with methylene chloride. The combined methylene chloride layers were washed with aqueous potassium bicarbonate solution and concentrated by vacuum distillation. 1-Butanol was added and the mixture was again concentrated by vacuum distillation. The resulting solution was stirred at approximately 20° C. during which time the product crystallized. The solids were collected by filtration, washed with 1-butanol to afford compound the title compound, which was isolated as a solvent wet cake and used directly in the next step. ¹HNMR (400 MHz, DMSO-d6): δ 7.69 (s, 1H), 7.24 (s, 1H); 4.23 (m, 2H), 3.94 (s, 3H), 3.78 (t)-3.65 (t) (2H), 2.51 (s, 3H), 2.30-2.08 (m, 2H) LC/MS Calcd for [M(Cl)+H]⁺ 288.1, found 288.0; Calcd for [M(Br)+H]⁺ 332.0, 334.0, found 331.9, 334.0.

### Preparation of 1-[5-methoxy-4-(3-morpholin-4-yl-propoxy)-2-nitro-phenyl]-ethanone

The solvent wet cake isolated in the previous step was dissolved in toluene. A solution of sodium iodide (67.9 kg) and potassium carbonate (83.4 kg) was added to this solution, followed by tetrabutylammonium bromide (9.92 kg) and morpholine (83.4 kg). The resulting 2 phase mixture was heated to approximately 85° C. for about 9 hours. The mixture was then cooled to ambient temperature. The organic layer was removed. The aqueous layer was back extracted with toluene. The combined toluene layers were washed sequentially with two portions of saturated aqueous sodium thiosulfate followed by two portions of water. The resulting solution of the title compound was used in the next step without further processing. ¹HNMR (400 MHz, DMSO-d6): δ 7.64 (s, 1H), 7.22 (s, 1H), 4.15 (t, 2H), 3.93 (s, 3H), 3.57 (t, 4H), 2.52 (s, 3H), 2.44-2.30 (m, 6H), 1.90 (quin, 2H); LC/MS Calcd for [M+H]⁺ 339.2, found 339.2.

### Preparation of 1-[2-amino-5-methoxy-4-(3-morpholin-4-yl-propoxy)-phenyl]-ethanone

The solution from the previous step was concentrated under reduced pressure to approximately half of the original volume. Ethanol and 10 percent Pd C (50 percent water wet, 5.02 kg) were added; the resulting slurry was heated to approximately 48° C. and an aqueous solution of formic acid (22.0 kg) and potassium formate (37.0 kg) was added. When the addition was complete and the reaction deemed complete by thin layer chromatography (TLC), water was added to dissolve the by-product salts. The mixture was filtered to remove the insoluble catalyst. The filtrate was concentrated under reduced pressure and toluene was added. The mixture was made basic (pH of about 10) by the addition of aqueous potassium carbonate. The toluene layer was separated and the aqueous layer was back extracted with toluene. The combined toluene phases were dried over anhydrous sodium sulfate. The drying agent was removed by filtration and the resulting solution was used in the next step without further processing. ¹HNMR (400 MHz, DMSO-d6): δ 7.11 (s, 1H), 7.01 (br s, 2H), 6.31 (s, 1H), 3.97 (t, 2H), 3.69 (s, 3H), 3.57 (t, 4H), 2.42 (s, 3H), 2.44-2.30 (m, 6H), 1.91 (quin, 2H LC/MS Calcd for [M+H]⁺ 309.2, found 309.1.

### Preparation of 6-methoxy-7-(3-morpholin-4-yl-propoxy)-quinolin-4-ol, sodium salt

A solution of sodium ethoxide (85.0 kg) in ethanol and ethyl formate (70.0 kg) was added to the solution from the previous step. The mixture was warmed to approximately 44° C. for about 3 hours. The reaction mixture was cooled to approximately 25° C. Methyl t-butyl ether (MTBE) was added which caused the product to precipitate. The product was collected by filtration and the cake was washed with MTBE and dried under reduced pressure at ambient temperature. The dried product was milled through a mesh screen to afford 60.2 kg of the title compound. ¹HNMR (400 MHz, DMSO-d6): δ 11.22 (br s, 1H), 8.61 (d, 1H), 7.55 (s, 1H), 7.54 (s, 1H), 7.17 (d, 1H), 4.29 (t, 2H), 3.99 (m, 2H), 3.96 (s, 3H), 3.84 (t, 2H), 3.50 (d, 2H), 3.30 (m, 2H), 3.11 (m, 2H), 2.35 (m, 2H), LC/MS Calcd for [M+H]⁺ 319.2, found 319.1.

### Preparation of 4-chlor-6-methoxy-7-(3 morpholin-4-yl)-quinoline

Phosphorous oxychloride (26.32 kg) was added to a solution of 6-methoxy-7-(3-morpholin-4-yl-propoxy)-quinolin-4-ol (5.00 kg) in acetonitrile that was heated to 50-55° C. When the addition was complete, the mixture was heated to reflux (approximately 82° C.) and held at that temperature, with stirring for approximately 18 hours at which time it was sampled for in process HPLC analysis. The reaction was considered complete when no more than 5 percent starting material remained. The reaction mixture was then cooled to 20-25° C. and filtered to remove solids. The filtrate was then concentrated to a residue. Acetronitrile was added and the resulting solution was concentrated to a residue. Methylene chloride was added to the residue and the resulting solution was quenched with a mixture of methylene chloride and aqueous ammonium hydroxide. The resulting 2 phase mixture was separated and the aqueous layer was back extracted with methylene chloride. The combined methylene chloride solutions were dried over anhydrous magnesium sulfate, filtered and concentrated to a solid. The solids were dried at 30-40° C. under reduced pressure to afford the title compound (1.480 kg). ¹HNMR (400 MHz, DMSO-d6): δ 8.61 (d, 1H), 7.56 (d, 1H), 7.45 (s, 1H), 7.38 (s, 1H), 4.21 (t, 2H), 3.97 (s, 3H), 3.58 (m, 2H), 2.50-2.30 (m, 6H), 1.97 (quin, 2H) LC/MS Calcd for [M+H]⁺ 458.2, found 458.0.

### Preparation of 4-(2-fluoro-4-nitro-phenoxy)-6-methoxy-7-(3-morpholin-4-yl propoxy)quinoline

A solution of 4-chloro-6-methoxy-7-(3 morpholin-4-yl)-quinoline (2.005 kg, 5.95 mol) and 2 fluoro-4-nitrophenol (1.169 kg, 7.44 mol) in 2,6-lutidine was heated to 140-145° C., with stirring, for approximately 2 hours, at which time it was sampled for in process HPLC analysis. The reaction was considered complete when less than 5 percent starting material remained. The reaction mixture was then cooled to approximately 75° C. and water was added. Potassium carbonate was added to the mixture, which was then stirred at ambient temperature overnight. The solids that precipitated were collected by filtration, washed with aqueous potassium carbonate, and dried at 55-60° C. under reduced pressure to afford the title compound (1.7 kg). ¹HNMR (400 MHz, DMSO-d6): δ 8.54 (d, 1H), 8.44 (dd, 1H), 8.18 (m, 1H), 7.60 (m, 1H), 7.43 (s, 1H), 7.42 (s, 1H), 6.75 (d, 1H), 4.19 (t, 2H), 3.90 (s, 3H), 3.56 (t, 4H), 2.44 (t, 2H), 2.36 (m, 4H), 1.96 (m, 2H). LC/MS Calcd for [M+H]⁺ 337.1, 339.1, found 337.0, 339.0.

### Preparation of 3-fluoro-4-[6-methoxy-7-(3-morpholin-4-yl-propoxy)-quinolin-4-yloxy]-phenylamine

A reactor containing 4-(2-fluoro-4-nitro-phenoxy)-6-methoxy-7-(3-morpholin-4-yl propoxy)quinoline (2.5 kg) and 10 percent palladium on carbon (50 percent water wet, 250 g) in a mixture of ethanol and water containing concentrated hydrochloric acid (1.5 L) was pressurized with hydrogen gas (approximately 40 psi). The mixture was stirred at ambient temperature. When the reaction was complete (typically 2 hours), as evidenced by in process HPLC analysis, the hydrogen was vented and the reactor inerted with argon. The reaction mixture was filtered through a bed of Celite® to remove the catalyst. Potassium carbonate was added to the filtrate until the pH of the solution was approximately 10. The resulting suspension was stirred at 20-25° C. for approximately 1 hour. The solids were collected by filtration, washed with water and dried at 50-60° C. under reduced pressure to afford the title compound (1.164 kg)._{—}¹H NMR (400 MHz, DMSO-d6): δ 8.45 (d, 1H), 7.51 (s, 1H), 7.38 (s, 1H), 7.08 (t, 1H), 6.55 (dd, 1H), 6.46 (dd, 1H), 6.39 (dd, 1H), 5.51 (br. s, 2H), 4.19 (t, 2H), 3.94 (s, 3H), 3.59 (t, 4H), 2.47 (t, 2H), 2.39 (m, 4H), 1.98 (m, 2H). LC/MS Calcd for [M+H]⁺ 428.2, found 428.1.

### Preparation of 1-(4-fluoro-phenylcarbamoyl)-cyclopropanecarboxylic acid

Triethylamine (7.78 kg) was added to a cooled (approximately 4° C.) solution of commercially available cyclopropane 1,1-dicarboxylic acid (9.95 kg) in THF, at a rate such that the batch temperature did not exceed 10° C. The solution was stirred for approximately 30 minutes and then thionyl chloride (9.14 kg) was added, keeping the batch temperature below 10° C. When the addition was complete, a solution of 4 fluoroaniline (9.4 kg) in THF was added at a rate such that the batch temperature did not exceed 10° C. The mixture was stirred for approximately 4 hours and then diluted with isopropyl acetate. The diluted solution was washed sequentially with aqueous sodium hydroxide, water, and aqueous sodium chloride. The organic solution was concentrated by vacuum distillation. Heptane was added to the concentrate. The resulting slurry was filtered by centrifugation and the solids were dried at approximately 35° C. under vacuum to afford the title compound (10.2 kg). NMR (400 MHz, DMSO-d6): δ 13.06 (br s, 1H), 10.58 (s, 1H), 7.65-7.60 (m, 2H), 7.18-7.12 (m, 2H), 1.41 (s, 4H), LC/MS Calcd for [M+H]⁺ 224.1, found 224.0.

### Preparation of 1-(4-fluoro-phenylcarbamoyl)-cyclopropanecarbonylchloride

Oxalyl chloride (291 mL) was added slowly to a cooled (approximately 5° C.) solution of 1-(4-fluoro-phenylcarbamoyl)-cyclopropanecarboxylic acid in THF at a rate such that the batch temperature did not exceed 10° C. When the addition was complete, the batch was allowed to warm to ambient temperature and held with stirring for approximately 2 hours, at which time in process HPLC analysis indicated the reaction was complete. The solution was used in the next step without further processing.

### Preparation of cyclopropane-1,1-dicarboxylic acid {3-fluoro-4-[6-methoxy-7-(3-morpholin-4-yl-propoxy)-quinolin-4-ylamino]phenyl}-amide-(4 fluorophenyl)-amide

The solution from the previous step was added to a mixture of 3-fluoro-4-[6-methoxy-7-(3-morpholin-4-yl-propoxy)-quinolin-4-yloxy]-phenylamine (1160 kg) and potassium carbonate (412.25 g) in THF and water at a rate such that the batch temperature was maintained at approximately 15-21° C. When the addition was complete, the batch was warmed to ambient temperature and held with stirring for approximately 1 hour, at which time in process HPLC analysis indicated the reaction was complete. Aqueous potassium carbonate solution and isopropyl acetate were added to the batch. The resulting 2-phase mixture was stirred and then the phases were allowed to separate. The aqueous phase was back extracted with isopropyl acetate. The combined isopropyl acetate layers were washed with water followed by aqueous sodium chloride and then slurried with a mixture of magnesium sulfate and activated carbon. The slurry was filtered over Celite® and the filtrate was concentrated to an oil at approximately 30° C. under vacuum to afford the title compound which was carried into the next step without further processing. ¹H NMR (400 MHz, DMSO-d6): δ 10.41 (s, 1H), 10.03 (s, 1H), 8.47 (d, 1H), 7.91 (dd, 1H), 7.65 (m, 2H), 7.53 (m, 2H), 7.42 (m, 2H), 7.16 (t, 2H), 6.41 (d, 1H), 4.20 (t, 2H), 3.95 (s, 3H), 3.59 (t, 4H), 2.47 (t, 2H), 2.39 (m, 4H), 1.98 (m, 2H), 1.47 (m, 4H). LC/MS Calcd for [M+H]⁺ 633.2, found 633.1.

### Preparation of the bisphosphate salt of cyclopropane-1,1-dicarboxylic acid {3-fluoro-4-[6-methoxy-7-(3-morpholin-4-yl-propoxy)-quinolin-4-ylamino]phenyl}-amide (4-fluorophenyl)-amide

Cyclopropane-1,1-dicarboxylic acid {3-fluoro-4-[6-methoxy-7-(3-morpholin-4-yl-propoxy)-quinolin-4-ylamino]phenyl}-amide-(4 fluoro phenyl)-amide from the previous step was dissolved in acetone and water. Phosphoric acid (85%, 372.48 g) was added at a rate such that the batch temperature did not exceed 30° C. The batch was maintained at approximately 15-30° C. with stirring for 1 hour during which time the product precipitated. The solids were collected by filtration, washed with acetone and dried at approximately 60° C. under vacuum to afford the title compound (1.533 kg). The title compound has a c-Met IC₅₀ value of less than 50 nM. The bisphosphate salt is not shown in scheme 1. ¹H NMR (400 MHz, DMSO-d6): (diphosphate) δ 10.41 (s, 1H), 10.02 (s, 1H), 8.48 (d, 1H), 7.93 (dd, 1H), 7.65 (m, 2H), 7.53 (d, 2H), 7.42 (m, 2H), 7.17 (m, 2H), 6.48 (d, 1H), 5.6 (br s, 6H), 4.24 (t, 2H), 3.95 (s, 3H), 3.69 (bs, 4H), 2.73 (bs, 6H), 2.09 (t, 2H), 1.48 (d, 4H).

### Procedure for Direct Coupling

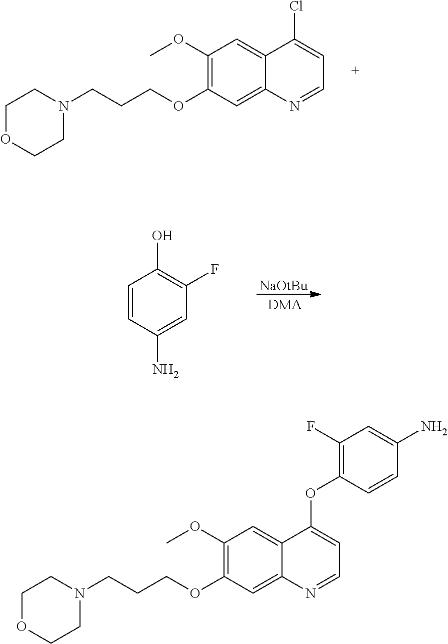

Solid sodium tert-butoxide (1.20 g; 12.5 mmol) was added to a suspension of the chloroquinoline (3.37 g; 10 mmol) in dimethylacetamide (35 mL), followed by solid 2-fluoro-4-hydroxyaniline. The dark green reaction mixture was heated at 95-100° C. for 18 hours. HPLC analysis showed approximately. 18 percent starting material remaining and approximately 79 percent product. The reaction mixture was cooled to below 50° C. and additional sodium tert-butoxide (300 mg; 3.125 mmol) and aniline (300 mg; 2.36 mmol) were added and heating at 95-100° C. was resumed. HPLC analysis after 18 h revealed less than 3% starting material remaining. The reaction was cooled to below 30° C., and ice water (50 mL) was added while maintaining the temperature below 30° C. After stirring for 1 hour at room temperature, the product was collected by filtration, washed with water (2×10 mL) and dried under vacuum on the filter funnel, to yield 4.11 g of the coupled product as a tan solid (96% yield; 89%, corrected for water content). ¹H NMR and MS: consistent with product; 97.8% LCAP; approximately 7 weight percent water by KF.

### Preparation of Compound 1 Hydrate Form

The hydrate of Compound 1 was prepared by adding 4.9614 g of Compound 1 and 50 mL of n-propanol to a 250 mL beaker. The suspension was heated to 90° C. with stirring via a magnetic stir bar at 200 rpm. After 2 hours, the solids were fully dissolved in an amber solution. At the 1 hour and 2 hour timepoints, 10 mL of n-propanol was added to account for evaporative effects and return the volume of the solution to 50 mL. The solution was then hot-filtered through a 1.6 μm glass fiber filter. The solution was then allowed to dry overnight in the beaker to a powder, which was then redissolved in 150 mL of a 1:1 mixture of acetone and water, and slurried overnight (16 hours) with a foil lid to prevent evaporation. The slurried solids were then collected by vacuum filtration. The final weight recovered was 3.7324 g (75% yield). This batch was stored at ambient conditions for several days prior to analysis.

Karl Fisher water content determinations were performed using a standard procedure. Water content was measured with a Brinkmann KF1V4 Metrohm 756 Coulometer equipped with a 703 Ti stirrer and using Hydranal Coulomat AG reagent. Samples were introduced into the vessel as solids. Approx 30-35 mg of sample was used per titration. A sample of crystalline Compound (I) prepared in Example 1.1.2 was measured in duplicate and was found to have an average water content be 2.5% w/w, with each replicate agreeing to within 0.1%.

A gravimetric vapor sorption (GVS) study was run using a standard procedure. Samples were run on a dynamic vapor sorption analyzer (Surface Measurement Systems) running DVSCFR software. Sample sizes were typically 10 mg. A moisture adsorption desorption isotherm was performed as outlined below. The standard isotherm experiment, performed at 25° C., is a two-cycle run, starting at 40% RH, increasing humidity to 90% RH, decreasing humidity to 0% RH, increasing humidity again to 90% RH, and finally decreasing humidity to 0% RH in 10% RH intervals. The crystalline Compound 1 prepared in Example 1.1.1 showed a 2.5% weight gain at 25° C. and 90% humidity. The GVS sorption and desorption curves showed evidence that the hydrate behaves as an isomorphic desolvate (Stephenson, G. A.; Groleau, E. G.; Kleeman, R. L.; Xu, W.; Rigsbee, D. R. J. Pharm. Sci. 1998, 87, 536-42).

The X-ray powder diffraction pattern of Compound 1 crystalline hydrate prepared above was acquired using a PANalytical X'Pert Pro diffractometer. The sample was gently flattened onto a zero-background silicon insert sample holder. A continuous 20 scan range of 2° to 50° was used with a CuKα radiation source and a generator power of 40 kV and 45 mA. A 20 step size of 0.017 degrees/step with a step time of 40.7 seconds was used. Samples were rotated at 30 rpm. Experiments were performed at room temperature and at ambient humidity. FIG. 1-B shows the XRPD pattern for N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide crystalline hydrate. The following peaks at an experimental °2θ+0.1°2θ were identified in the XRPD pattern: 6.6, 9.0, 10.2, 12.0, 12.2, 13.1, 13.3, 14.6, 15.6, 16.2, 17.0, 17.1, 17.4, 18.2, 18.4, 18.7, 20.0, 20.3, 20.8, 21.7, 22.1, 23.1, 23.4, 23.8, 24.2, 24.5, 25.0. Only peaks below 25° 2θ are given as these are generally preferred for the identification of crystalline pharmaceutical forms. The entire list of peaks, or a subset thereof, may be sufficient to characterize the hydrate of Compound 1.

DSC thermograms were acquired using a TA Instruments Q2000 differential scanning calorimeter. A sample mass of 2.1500 mg of Compound 1 crystalline hydrate was weighed out directly into an aluminum DSC pan. The pan was sealed by applying pressure by hand and pushing each part the pan together (also known as a loose lid configuration). The temperature was ramped from 25° C. to 225° C. at 10° C./minute. A peak melting temperature of 137.4° C. and a heat flow of 44.2 J/g was measured for the melting endotherm. After the melting event, recrystallization occurs to an anhydrous form, which then melts at 194.1° C.

TGA thermograms were acquired using a TA Instruments Q500 Thermogravimetric Analyzer. The sample pan was tared, and 9.9760 milligrams of Compound (I) crystalline hydrate was placed in the pan. The temperature was ramped from 25° C. to 300° C. at 10° C./minute. A weight loss of 2.97% was observed up to 160° C., with an additional weight loss beyond 200° C. from decomposition.

Preparation of Compound 1 Crystalline Hydrate with Different Hydration States.

Five 150 mg aliquots were taken from the crystalline hydrate batch prepared above and were placed in 10 mL screw-top vials. With the vial tops removed, these aliquots were each stored in chambers with desiccant (Dri-Rite®, tricalcium silicate, RH 2-3%), saturated lithium bromide (6% RH), saturated lithium chloride (11% RH), saturated magnesium chloride (33% RH), and saturated sodium chloride (75% RH). The samples were removed after 2 weeks and immediately sealed with a cap for analysis and characterized.

### Case Studies

The MET and VEGF signaling pathways appear to play important roles in osteoblast and osteoclast function. Strong immunohistochemical staining of MET has been observed in both cell types in developing bone. HGF and MET are expressed by osteoblasts and osteoclasts in vitro and mediate cellular responses such as proliferation, migration, and expression of ALP. Secretion of HGF by osteoblasts has been proposed as a key factor in osteoblast/osteoclast coupling, and in the development of bone metastases by tumor cells that express MET. Osteoblasts and osteoclasts also express VEGF and its receptors, and VEGF signaling in these cells is involved in potential autocrine and/or paracrine feedback mechanisms regulating cell migration, differentiation, and survival.

Bone metastases are present in 90% of patients with castration-resistant prostate cancer (CRPC), causing significant morbidity and mortality. Activation of the MET and VEGFR signaling pathways is implicated in the development of bone metastases in CRPC. Three metastatic CRPC patients treated with Compound 1, an inhibitor of MET and VEGFR, had dramatic responses with near complete resolution of bone lesions, marked reduction in bone pain and total serum alkaline phosphatase (tALP) levels, and reduction in measurable disease. These results indicate that dual modulation of the MET and VEGFR signaling pathways is a useful therapeutic approach for treating CRPC.

Compound 1 is an orally bioavailable multitargeted tyrosine kinase inhibitor with potent activity against MET and VEGFR. Compound 1 suppresses MET and VEGFR signaling, rapidly induces apoptosis of endothelial cells and tumor cells, and causes tumor regression in xenograft tumor models. Compound 1 also significantly reduces tumor invasiveness and metastasis and substantially improves overall survival in a murine pancreatic neuroendocrine tumor model.

Based on the target rationale, Compound 1 will be administered as up to a 250 mg dose to patients with CRPC. The compound is expected to lead to a decrease in uptake of radiotracer on bone scan upon treatment with Compound 1. The findings are expected to be acre accompanied by substantial reductions in bone pain as well as evidence of response or stabilization in soft tissue lesions during therapy with Compound 1. The onset of the effect is expected to be rapid.

Uptake of radiotracer in bone depends on both local blood flow and osteoblastic activity, both of which may be pathologically modulated by the tumor cells associated with the bone lesion. Resolving uptake may therefore be attributable to either interruption of local blood flow, direct modulation of osteoblastic activity, a direct effect on the tumor cells in bone, or a combination of these processes. However, decreased uptake on bone scan in men with CRPC has only been rarely noted with VEGF/VEGFR targeted therapy, despite numerous trials with such agents. Similarly, observations of decreased uptake on bone scan in CRPC patients have only been reported rarely for abiraterone, which targets the cancer cells directly, and for dasatinib, which targets both cancer cells and osteoclasts. Thus, targeting angiogenesis alone, or selectively targeting the tumor cells and/or osteoclasts, has not resulted in effects similar to those observed in the patients treated with Compound 1.

The potential results are expected to indicate a potential critical role for the MET and VEGF signaling pathways in the progression of CRPC and point to the promise that simultaneously targeting these pathways may have in reducing morbidity and mortality in this patient population

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1. A method for treating bone cancer, prostate cancer, or bone cancer associated with prostate cancer, comprising administering a compound that dually modulates MET and VEGF to a patient in need of such treatment, wherein the compound that dually modulates MET and VEGF is a compound of Formula I is the compound of Formula I: 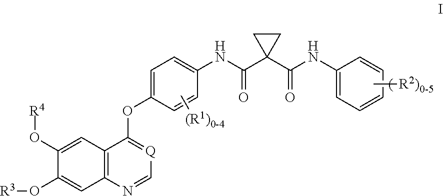
or a pharmaceutically acceptable salt thereof, wherein:
R¹ is halo;
R² is halo;
R³ is (C₁-C₆)alkyl substituted with heterocycloalkyl;
R⁴ is (C₁-C₆)alkyl; and
Q is CH or N.

2. The method of claim 1, wherein the bone cancer is osteoblastic bone metastases.

3. The method of claim 1, wherein the prostate cancer is CRPC.

4. (canceled)

5. The method of claim 1, wherein the dual MET and VEGF modulator is a compound of Formula Ia 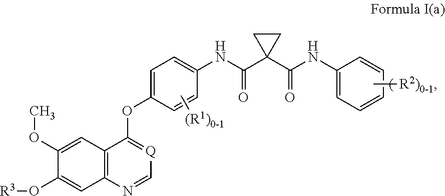
or a pharmaceutically acceptable salt thereof, wherein:
R¹ is halo;
R² is halo;
R³ is (C₁-C₆)alkyl substituted with heterocycloalkyl; and
Q is CH or N.

6. The method of claim 1, wherein Q is CH. or a pharmaceutically acceptable salt thereof, wherein:
R¹ is halo;
R² is halo; and
R³ is (C₁-C₆)alkyl substituted with heterocycloalkyl.

7. The method of claim 1, wherein the compound of Formula I is Compound 1.

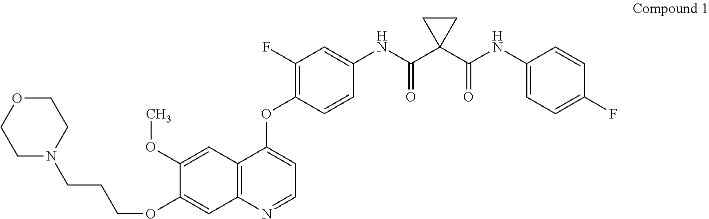 [SMILES: COc1cc2c(Oc3ccc(NC(=O)C4(C(=O)Nc5ccc(F)cc5)CC4)cc3F)ccnc2cc1OCCCN1CCOCC1]

8. The method of claim 7, wherein Compound I is in the crystalline hydrate form.

9. The method of claim 1 wherein the compound of Formula I, I(a), or I(b), Compound 1 or a pharmaceutically acceptable salt thereof, is administered as a pharmaceutical composition additionally comprising a pharmaceutically acceptable carrier, excipient, or diluent.

10. A method of a method for treating osteoblastic bone metastases associated with CRPC, comprising administering a pharmaceutical formulation comprising Compound of Formula I or the malate salt of Compound of Formula I or another pharmaceutically acceptable salt of Compound of Formula I, to a patient in need of such treatment.

11. A method for ameliorating abnormal deposition of unstructured bone accompanied, increased skeletal fractures, spinal cord compression, and severe bone pain of osteoblastic bone metastases, comprising administering to a patient in need of such treatment a therapeutically effective amount of a compound of Formula I in any of the embodiments disclosed herein.
